(19) United States
(12) Patent Application Publication
(10) Pub. No.: US 2017/0112947 A1
(43) Pub. Date: Apr. 27, 2017

(54) COMPOSITION FOR USE IN IMAGING

(71) Applicant: The Trustees of Columbia University in the City of New York, New York, NY (US)

(72) Inventor: Mulugeta S. Abebe, New York, NY (US)

(21) Appl. No.: 15/315,258

(22) PCT Filed: Jun. 5, 2015
(86) PCT No.: PCT/US15/34360

§ 371 Date: Nov. 30, 2016

Related U.S. Application Data
(60) Provisional application No. 62/007,990, filed on Jun. 5, 2014.

(51) Int. Cl. A61K 49/00; A61K 49/04; A61K 47/44; A61K 47/22; A61K 47/32; G01R 33/28; A61K 47/02; A61K 47/42; A61B 5/055; A61B 6/03; A61B 6/00; A61K 49/06; A61K 47/46

(52) U.S. Cl. CPC A61K 49/0002; A61K 49/06; A61K 49/04; A61K 47/44; A61K 47/22; A61K 47/32; A61K 47/46; A61K 47/02; A61K 47/42; A61B 5/055; A61B 6/03; A61B 6/481; G01R 33/282

(57) ABSTRACT

The present invention is for a novel composition for imaging using magnetic resonance imaging (MRI), x-rays, and x-ray computed tomography (CT). The composition shows up as an image on all type of MRIs, x-rays and CT scans, and is made of natural ingredients suitable for topical use. The composition is versatile and flexible. The invention is also to a method of using the composition to visualize surfaces and structures that are not visible on scan images, and to more precisely localize internal structures from MRI, x-rays and CT images.

### CROSS-REFERENCE TO RELATED APPLICATIONS

The present application claims priority to U.S. Patent Application Ser. No. 62/007,990, filed Jun. 5, 2014, which is hereby incorporated by reference.

This invention was made with government support under 1 R01 EY014978-06, 2T32MH015174-35:38, and the NARSAD Young Investigator Award awarded by the National Institutes of Health. The government has certain rights in the invention

### FIELD OF THE INVENTION

This invention is a composition useful for visualizing external surfaces of the body, e.g., biological tissue, and naturally and non-naturally occurring structures outside the body, e.g., electrodes, which are usually invisible in imaging procedures. The invention is also a method of using the composition. The compositions and methods of the invention can be used in any imaging procedure including but not limited to, magnetic resonance imaging (MRI), x-ray computed tomography (CT), and x-rays.

### BACKGROUND OF THE INVENTION

Medical and engineering practices constantly employ the use of devices and methods for visualizing internal structures in humans, animals, and machines. Three very well established methods, magnetic resonance imaging (MRI), computed tomography imaging (CT), and x-ray imaging, are in wide use and have improved our lives in many ways. MR1 methods continue to be the choice imaging tools for several practical purposes, most particularly, medical imaging. According to the European Forum for Magnetic Resonance Research and Application, there are 25,000 MRI machines worldwide, 10,000 of which are in the United States.

Numerous research and clinical interventions, such as targeted drug deliveries and surgeries, require accurate localization of various body parts. For example, in brain surgery, both in human and non-human primates, access to an area of interest is often gained by making large craniotomies using typical stereotactic procedures that rely upon external cranial landmarks and standardize atlases (Saunders et al. 1990). Even though the overall organization and relative location with respect to each other stays the same between individuals, there is variability in pattern of brain area folding, shape and size of cortical areas and relative locations (Van Essen et al. 1998). It is very common to find errors in subjective guesses of location of a brain structure from skull topography alone. Moreover, as standardized atlases are generally used for localization of subcortical neural regions (Saunders et al. 1990), problems still arise from such poorly informed assumptions of the location of underlying brain structures, and it is common to make a misplaced craniotomy.

Since MRI is a non-invasive method that capitalizes on the complex mosaic across the cortical sheet (Van Essen et al. 1998), it is possible to solve these problems by taking individual MRIs pre-surgery, mapping the structures of interest to an external marker, and using the established coordinates during the surgery. However, one of the major issues with MRI is how to externally localize a given body part or structure invisible to the naked eye on an image, and correctly establish the relationship between body landmarks and MRI scans. Thus, there is a need for a method that allows invisible surfaces and structures to be visible on scanned images in order to a reconstruct the areas of interest and describe the relationships within a reasonably acceptable mathematical error.

While recently a new method of expressing relationships between surface markers, such as tattoos on head skin and underlying major brain structures has been developed, (Semework 2010), this method has limitations. Also, there are contrast agents on the market, such as gadolinium. However, these are for injection, not external use. Even if these chemicals could be used externally, they do not show up as the same color in scans, and are very expensive. Another common practice is the use of vitamin E tablets for marking. However, for a variety of reasons, vitamin E tablets are not suitable for wide use.

The current invention solves these problems with the development and use of a novel composition that can be seen in all types of magnetic resonance images as well as in computed tomography scans and x-rays.

### SUMMARY OF THE INVENTION

The current invention is based upon the surprising discovery that a mixture of vitamin E, a solvent such as water or povidone-iodine, and fat can be visualized on all types of magnetic resonance images, including T1, T2, and FLAIR, as well as computed tomography scans and x-rays. Thus, the compound or composition of the invention, which can be applied to any surface, can be used for visualizing surfaces, biological tissue, structures, and organs that would not necessarily be visible on an image produced by an imaging procedure. The composition can be used for precisely localizing naturally occurring and non-naturally occurring structures in the body of a subject undergoing any imaging procedure including but not limited to, magnetic resonance imaging, x-ray computed tomography and x-rays, by providing an external reference point for an internal structure.

Thus, one embodiment of the present invention is the compound or composition. In the most preferred embodiment, the composition comprises vitamin E, a solvent such as water or povidone-iodine, and fat. The composition is suitable for topical application, such that it can be applied to the external and internal biological tissue of the subject, most likely the skin and dura.

The compound or composition can further comprise other ingredients including oil, an emulsifying agent, food color, an iron containing substance, a manganese containing substance, an opaque substance such as calcium carbonate or hydroxyapatite, i.e., calcium phosphate (especially when the composition is used in x-rays and CT scans), protein powder, and preservatives.

The composition can be made with different viscosities or consistencies, odors and colors. The composition can take many forms including but not limited to, a paste, a solution, a tablet, and a capsule.

The composition can be applied to external biological tissue, such as skin, to internal biological tissue or organs, such as dura, and to non-naturally occurring structures, such as electrodes.

The composition can also be placed into a device or vessel, such as tubing or a capsule, which can be placed on to, contacted with, or attached to an external surface, i.e., biological tissue, or structure on the subject, or placed internally onto or into a surface or structure of the subject.

The composition can also be loaded into machines for real time visualization that currently uses other compositions, such as gadolinium as a marker. These machines include those used for orienting during surgery and performing surgery such as those used for laparoscopy.

Further embodiments of the present invention are methods of using the compound or composition to visualize structures, organs, surfaces, or biological tissue on an image that would otherwise be invisible on the image.

A further embodiment of the present invention is a method of using the compound or composition for localizing an internal structure in a subject undergoing an imaging procedure to an external or internal surface or structure, which is normally invisible on an image from an imaging procedure. In this method, the composition is applied to external or internal surface or structure on or in the subject, such as biological tissue or an electrode, in order to visualize the external or internal surface or structure on the image, and the imaging procedure is performed on the subject. The external or internal surface or structure can have a known spatial relationship to the internal structure that is to be localized. After the subject undergoes the imaging procedure, the composition applied to the external or internal surface or structure is visualized on the resulting image, and the internal structure in the subject is localized to the external or internal surface or structure by the location of the composition on the image. The external or internal surface or structure can be naturally or non-naturally occurring. The internal structure to be localized can be naturally or non-naturally occurring.

Yet a further embodiment of the present invention is a method of using the compound or composition for localizing an internal structure in a subject undergoing an imaging procedure, where the composition placed into a device or vessel. The resulting device or vessel is placed on, contacted with, or attached to an external or internal surface or structure on or in the subject, and the imaging procedure is performed on the subject. The external or internal surface or structure can have a known spatial relationship to the internal structure that is to be localized. After the subject undergoes the imaging procedure, the composition is visualized on the resulting image, and the internal structure in the subject is localized to the device or vessel by the location of the composition on the image. The device or vessel can be in many forms including but not limited to, a tube or a capsule made of a sterile agent. The external or internal surface or structure can be naturally or non-naturally occurring. The internal structure to be localized can be naturally or non-naturally occurring.

### BRIEF DESCRIPTION OF THE FIGURES

For the purpose of illustrating the invention, there are depicted in drawings certain embodiments of the invention. However, the invention is not limited to the precise arrangements and instrumentalities of the embodiments depicted in the drawings.

FIG. 1 shows the result of three types of MRI scans, T1 (left panel), T2 (middle panel), and T2 FLAIR (right panel), using the composition of the present invention (“Mix”), the composition of the present invention plus gadolinium (“Mix+Gd”), olive oil (“Ingredient #1”), purified butter (“Ingredient #2”), water (“Future Ingredient #3”), gadolinium (“Gd”), toothpaste, toothpaste plus gadolinium (“Toothpaste+Gd”), and vitamin E tablet (“Vitamin E”).

FIG. 2A are MRI scans of a monkey brain using the composition of the present invention and four different types of MRI scan, BRAVO, T2 FSE, T1 FLAIR and T2 FLAIR. The composition of the current invention is shown by the thin layer of white and the dark arrows. It is on top of dental acrylic indicated by the opaque mound and white arrows. FIG. 2B is a photograph showing the actual dental acrylic implant in the monkey, the orientation marker, and the composition placement.

FIG. 3 depicts a model of a plastic helmet with a network of vinyl tubing filled with the composition of the present invention.

FIG. 4 are images of nine different types of MRI scans performed with the helmet shown in FIG. 3.

FIG. 5 shows the MRI scan results for different sequence pulses (BRAVO, T1 FLAIR, T2 FLAIR. MRA, and MRV) using the composition of the present invention ((A)—formulation 1 with dental acrylic, (B)— formulation 2 with dental acrylic, (D)—formulation 1 alone, and (E)—formulation 2 alone) and dental acrylic alone (C) as compared to scans of fixed brains.

FIG. 6 depicts results of BRAVO (bottom panels) and T1-weighted (top panels) MRIs acquired with fiducial markers fixed to a primate headpost. In the left-hand panels, where no compound was on the markers, the markers were not visible. In the right-hand panels, the markers were coated with the compound of the invention, and were visible in both scans (circled).

FIG. 7 depicts the results of x-rays using the composition of the present invention ((B)— formulation 1 with dental acrylic, (C)— formulation 2 with dental acrylic, (E) and (I)—formulation 1 alone, and (F) and (J)—formulation 2 alone) and dental acrylic alone (D) as compared to scans of fixed brains, stainless steel guide tube, 2 mm diameter (A), a 75 μM thick microelectrode (G), and a 250 μm thick microelectrode (H). The figure shows the same scan with increasing contrast from top to bottom panel.

FIG. 8 depicts the results of CT scans using the composition of the present invention ((B)— formulation 1 with dental acrylic, (C)— formulation 2 with dental acrylic, (E) and (I)— formulation 1 alone, and (F) and (J)—formulation 2 alone) and dental acrylic alone (D) as compared to scans of fixed brains, stainless steel guide tube, 2 mm diameter (A), a 75 μm thick microelectrode (G), and a 250 μm thick microelectrode (H). The figure shows the same scan with increasing contrast from top to bottom panel.

FIG. 9 depicts the results of high (upper image) and low dose (lower image) of CT scans using the composition of the present invention ((B)— formulation 1 with dental acrylic, (C)— formulation 2 with dental acrylic, (G)—formulation 1 alone, and (H)— formulation 2 alone) and dental acrylic alone (D) as compared to scans of fixed brains, stainless steel guide tube (A), a 75 μm thick microelectrode (E), and a 250 μm thick microelectrode (F).

### DETAILED DESCRIPTION OF THE INVENTION

The current invention is a novel compound or composition comprising vitamin E, a solvent such as water or povidone-iodine, and fat that can be visualized on all types of magnetic resonance images, computed tomography scans and x-rays. The current invention is also a method of using the composition to visualize surfaces, biological tissue, structures, and organs that would not necessarily be visible in an image produced by an imaging procedure, and to precisely localize structures in the body of a subject undergoing any imaging procedure including but not limited to, magnetic resonance imaging, x-ray computed tomography and x-rays, by providing an external reference point for an internal structure.

### DEFINITIONS

The terms used in this specification generally have their ordinary meanings in the art, within the context of this invention and the specific context where each term is used. Certain terms are discussed below, or elsewhere in the specification, to provide additional guidance to the practitioner in describing the methods of the invention and how to use them. Moreover, it will be appreciated that the same thing can be said in more than one way. Consequently, alternative language and synonyms may be used for any one or more of the terms discussed herein, nor is any special significance to be placed upon whether or not a term is elaborated or discussed herein. Synonyms for certain terms are provided. A recital of one or more synonyms does not exclude the use of the other synonyms. The use of examples anywhere in the specification, including examples of any terms discussed herein, is illustrative only, and in no way limits the scope and meaning of the invention or any exemplified term. Likewise, the invention is not limited to its preferred embodiments.

The terms “compound” and “composition” are used interchangeable in this application.

The term “subject” as used in this application means an animal with an immune system such as avians and mammals. Mammals include canines, felines, rodents, bovine, equines, porcines, ovines, and primates. Avians include, but are not limited to, fowls, songbirds, and raptors. Thus, the invention can be used in veterinary medicine, e.g., to treat companion animals, farm animals, laboratory animals in zoological parks, and animals in the wild. The invention can also be used on research animals. The invention is particularly desirable for human medical applications.

The term “patient” as used in this application means a human subject.

The term “about” or “approximately” means within an acceptable error range for the particular value as determined by one of ordinary skill in the art, which will depend in part on how the value is measured or determined, i.e., the limitations of the measurement system, i.e., the degree of precision required for a particular purpose, such as a pharmaceutical formulation. For example, “about” can mean within 1 or more than 1 standard deviations, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated, the term “about” meaning within an acceptable error range for the particular value should be assumed.

The term “internal” as used in this application means within the body of the subject or patient.

The term “external” as used in this application means outside the body of the subject or the patient.

The term “structure” as used in this application means: a naturally occurring structure, such as an organ including but not limited to, the brain, heart, kidneys, liver, lungs, stomach, pancreas, gall bladder, reproductive organs, intestines, bones, muscles, tendons, cartilage, and ligaments; or biological tissue including but not limited to, skin, epidermis, dura, dermis, adipose, musculoskeletal, and vascular; or a non-naturally occurring structure.

The term “non-naturally occurring structure” as used in the application means structures that are not naturally occurring in the body of a subject or patient, and that may have been implanted internally into the body of the subject or patient, or contacted or placed or implanted on the external surface of the body of the subject or patient including but not limited to, devices such as pacemakers and monitors, wires and electrodes, grafts and prosthetics, markers, posts, and tape.

The term “topical” as used in this application means suitable to be applied to any surface of the body.

The term “imaging procedure” as used in the application means a technique or process, either known or developed in the future, to create a visual representation of the interior of an object or the body of a subject or a patient. “Medical imaging” refers to imaging of the interior of a body for clinical analysis and medical intervention. Medical imaging seeks to reveal internal structures hidden by the skin and bones, as well as to diagnose and treat disease.

### The Imaging Compound/Composition

MRI images as well as CT scans and x-rays, are highly valuable tools for research and diagnosis. However, their efficacy to date has been limited by the fact there are surfaces and structures that cannot be imaged, i.e., cannot be seen on the scan. These are usually structures, either naturally or non-naturally occurring, that of are interest with regard to imaging, especially in relation to something near them. For example, if a plastic surgeon wants to determine how a patient's cheek and nose structure relates to the underlying bone before surgery, he would have to take several scans and change the parameters to accent the specific targets, e.g., bone as opposed to fat. If there was a technique to mark the skin that can be seen in a scan and showed him where the bone is in relation to the skin, then he could take one scan. Moreover, one of the limitations of x-rays is that it is still difficult to easily image and know the exact shape of objects that are relatively transparent, for instance skin compared to bone. Often because of this limitation, additional scans, such as an MRI, needs to be performed to further analyze and understand the object, i.e., body part, being studied or examined. The use of the imaging composition of the present invention alleviates the need for several scans and/or additional types of scans to further analyze the object (body part) under study.

The current method for marking in these types of scans uses vitamin E tablets. However, this method poses three problems for a wider use.

1. Vitamin E does not show up equally and clearly in several MRI sequences, such as T1, T2 and FLAIR.

2. The tablets are not flexible and sizable to affix to different external body parts which are not always straight or easily accessible.

3. Not all external body parts that are accessible and straight allow reliable contact (stickiness) with the tablets.

The problems with vitamin E tablets could be solved by a flexible and safe substance which can be used with all MRI types, CT scans and x-rays. The composition of current invention provides these solutions. Because it is made from natural ingredients, it is safe for topical use and can be manufactured as a sterile product that can easily and safely be applied to exposed body parts including to exposed brain or dura. It can be made with different viscosities (consistencies), colors, and odors for different uses. It can also be mixed with hardening materials such as acrylic for different uses, including medical applications and industrial and engineering applications. Also because vitamin E is a preservative, the composition is stable and can be used months after being prepared.

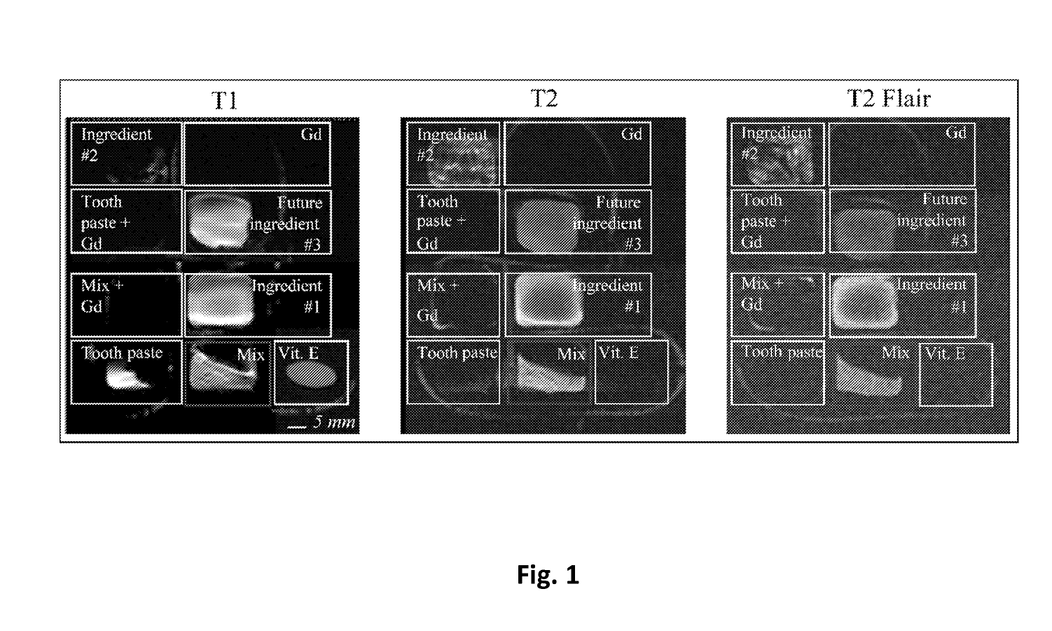

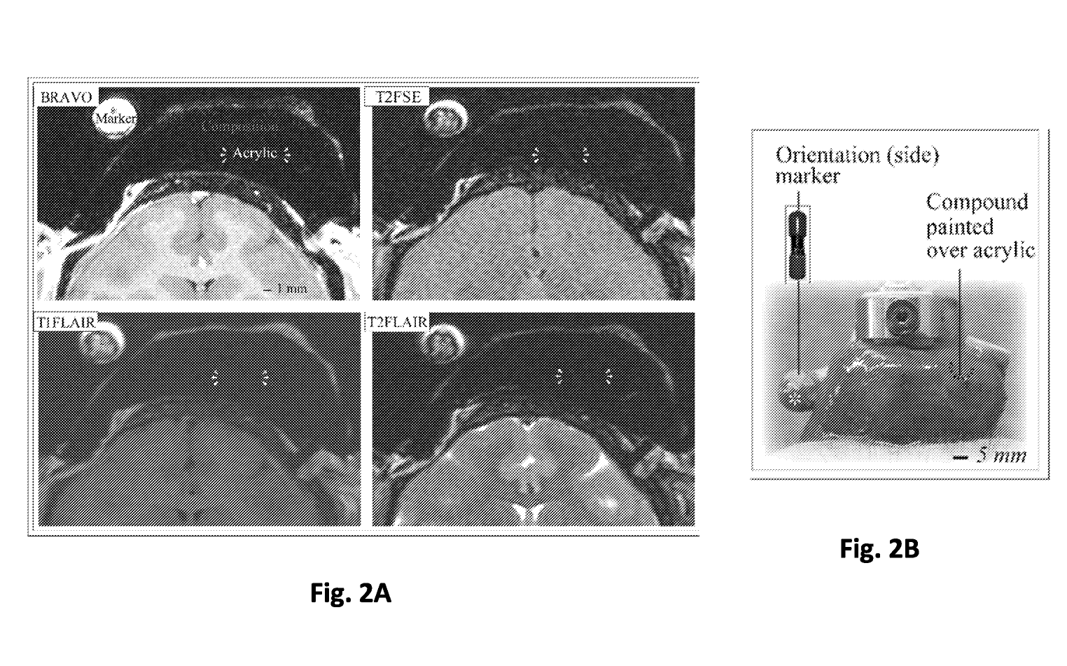

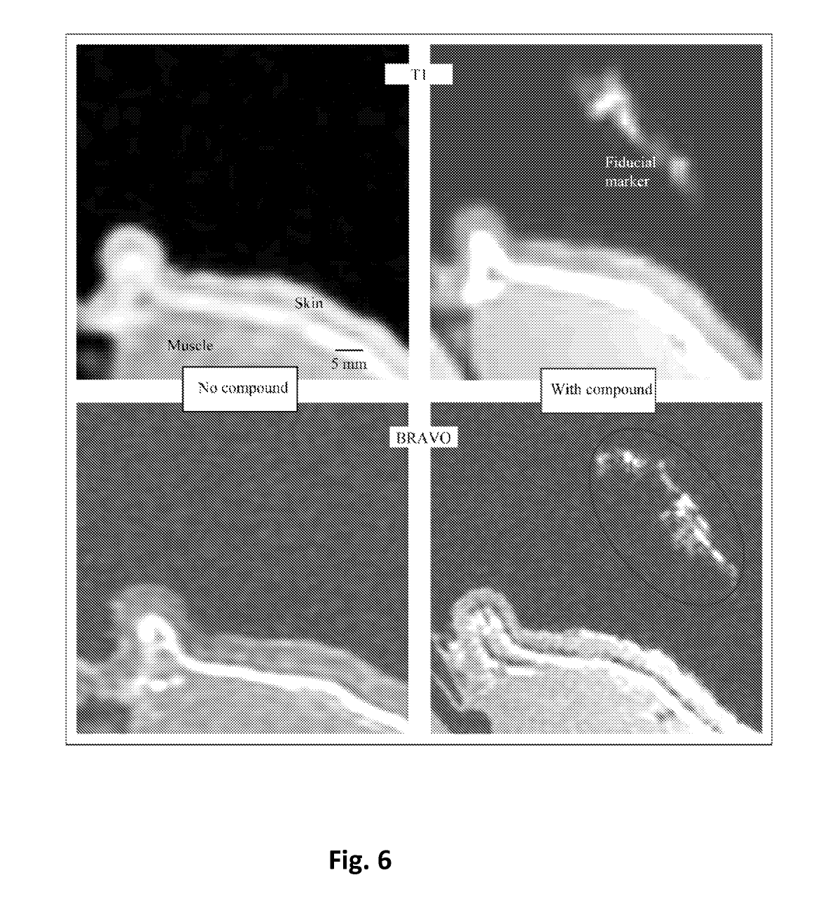

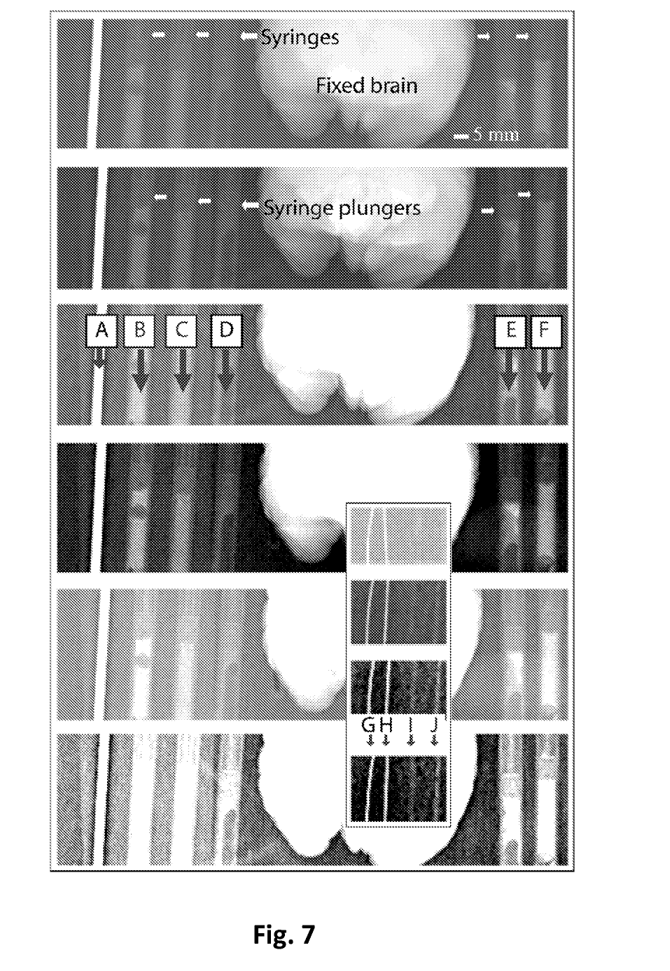

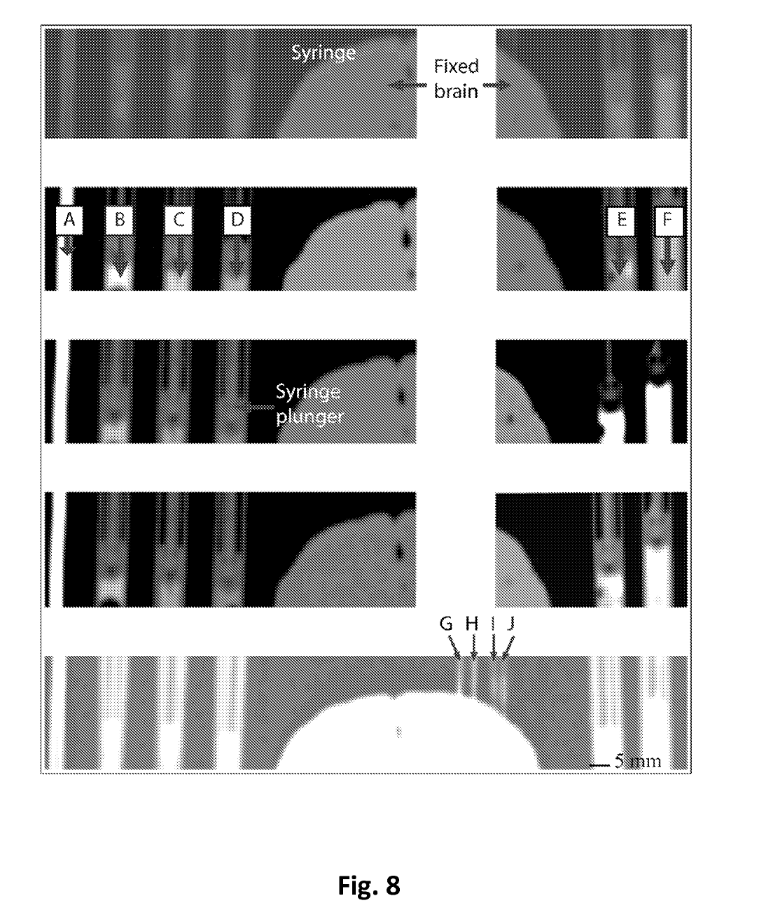

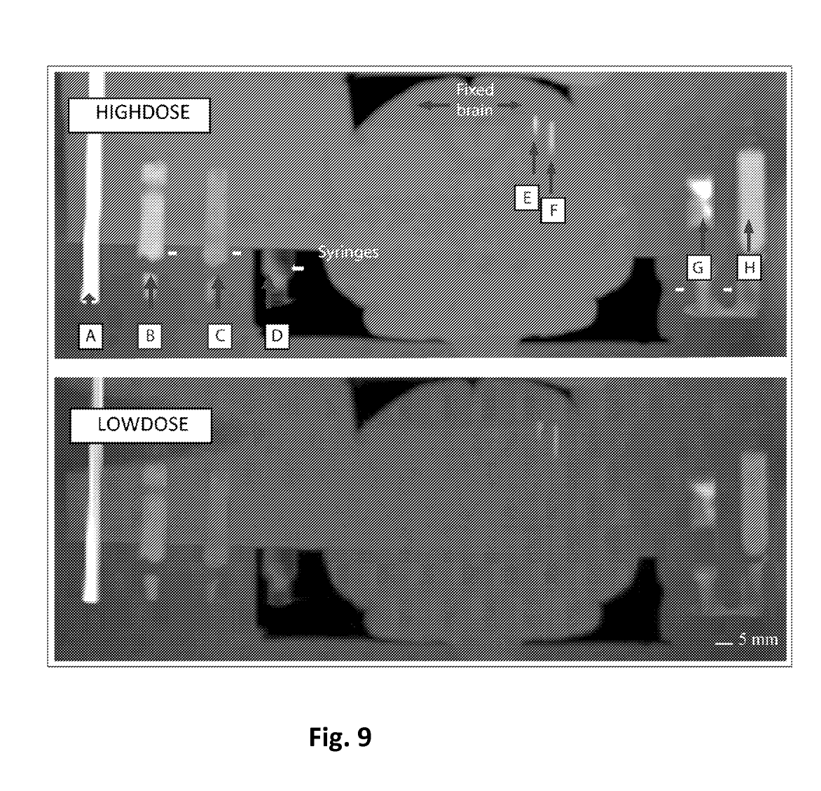

As shown in the Examples, the compound of the present invention solves all of the problems currently found with vitamin E tablets. The composition shows a clear and equal image on all types of MRI images, including the most often used, T1, T2, and FLAIR (Examples 2-6; FIGS. 1-6), as well as CT scans (Example 8; FIGS. 8 and 9) and x-rays (Example 7; FIG. 7). The composition is flexible and can be formed into many consistencies and shapes (Examples 3-8; FIGS. 2-9). Lastly, the composition can adhere to various body shapes such as heads, and various materials, both natural and non-natural (Examples 3 and 6; FIGS. 2 and 6).

Additionally, because vitamin E tablets are fat soluble, they cannot be mixed with or dissolved in water and thus, are not useful for topical application. The composition of the present invention and its method of use overcome this issue. Moreover, the fact that oil as well as vitamin E does not dissolve in water is exploited in the current invention because different MRI sequences pick up and show different substances.

The compound or composition of the present invention comprises:

A solvent, preferably water or povidone-iodine;

Fat; and

Vitamin E.

These ingredients are in the compound or composition, in amounts ranging from (percent by volume): a main solvent of water or povidone-iodine (PVP-I) (Betadine®)—about 25-75%, preferred about 25%-50%, and most preferred about 30-45%; fat—about 15-60%, preferred about 15-40%, more preferred about 20-30%, and most preferred about 5-10%; and vitamin E—about 15-60%, preferred about 15-40%, more preferred about 20-30%, and most preferred, 15-20%.

Vitamin E is preferentially used in oil form.

While any water can be used in the composition, distilled, double distilled, deionized, or purified water is preferred. Clinical grade purified water which can be obtained from companies such as ELGA are most preferred.

Fats that can be used in the composition of the present invention include but are not limited to, purified butter, vegetable shortening, coconut oil, and lard.

The compound or composition can also optionally comprise additional components such as olive oil, and additional materials specific to the particular use. In such embodiments, the ingredients of the compound would be in amounts ranging from (percent by volume): 1. a main solvent of water or povidone-iodine (PVP-I) (Betadine®)—about 30-45%; 2. vitamin E—about 15-20%; 3. fat—about 5-40%; 4. oil—about 5-10%; and 5. other materials specific to the use. The percentage of these last ingredients would be modified based upon the use and consistency of the composition, and can be as high as a total of about 45%. When needed, the composition can comprise (percent by volume):

| | |
|---|---|
| Emulsifying agent | about 3-10% |
| Food color | about 1% |
| Iron containing substance | about 1-3% |
| Manganese containing substance | about 1-3% |
| Hydroxyapatite | about 10-20% |
| or calcium carbonate | |
| Pure protein powder | about 10-15% |

Preservatives

Oils that can be used in the composition include but are not limited to, olive, vegetable, and nut oils.

Emulsifying agents that can be used in the composition include but are not limited to, egg yolk, borax, beeswax, and lecithin.

Iron containing substances that can be used in the composition include but are not limited to, thyme.

Manganese containing substances that can be used in the composition include but are not limited to, clove.

Hydroxyapatite (i.e., calcium phosphate) or calcium carbonate can be included in the composition especially when the composition is being used for x-ray or CT images.

Pure protein powder can be used to obtain a desired consistency. Pure protein powder that can be used in the composition include but are not limited to, whey protein and ground grains with high protein content such as wheat rice, amaranth, bulgar, and quinoa.

Preservatives include but are not limited to, salt and lemon.

While the preferred color of the composition of the present invention is white, with the use of food coloring the composition can be made any color desired by the skilled practitioner. One advantage of the composition of the present invention is that it shows as the same color in different scan types, while most products on the market such as contrast agents are oppositely colored. Tests using MRI and CT scans has shown that the composition has well delineated radio density.

The composition of the present invention can be used and shaped into many forms.

The most common form of the composition would be a paste. Such a paste could be applied to skin at any surface of a subject, including one with hair, and would adhere to the skin for the entirety of the scan. The paste can also be applied to internal biological tissue such as dura. The paste can also be applied to non-naturally occurring surfaces and structures such as tape and electrodes.

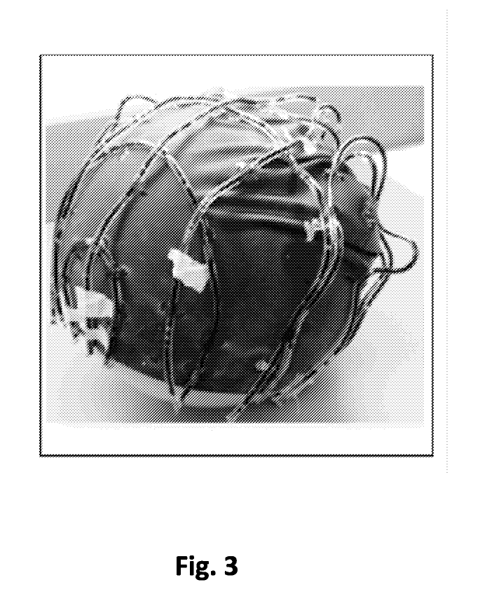

The present composition can also be made into a solution which can be poured or filled or placed into a vessel. The percentages of the composition need only be varied as set forth above to obtain this form. An example of this is placing the composition into tubes or tube-like structures, which are then placed on or attached to or contacted with the external surface of the body in relation to the internal body structure (see Example 3 and FIG. 3).

The composition can also be loaded into machines for real time visualization that currently use other compositions such as gadolinium as a marker. These machines include those used for orienting during surgery and performing surgery such as those used for laparoscopy.

The composition of the present invention can also be placed in mold such as letters or numbers to be place on the body for further reference during MRI or other imaging procedures.

The composition of the present invention can also be made into tablet form.

The composition can also be encapsulated in a sterile agent.

The composition of the present invention, with the range of amounts of ingredients, set forth above, is very effective in allowing the visualization of surfaces and structures that are normally invisible in all scans, including MRI, CT, and x-rays. It is within the skill of those in the art to alter and adjust the amounts of the specific components of the composition, depending on the use, the consistency and form, e.g, paste, liquid, tablet, or capsule, the type of imaging procedure being used, and other variables. For example, T2 MRI is used to visualize and quantify water, thus, when using the composition for this sequence, the composition should contain more water and less vitamin E and fat. When the composition is used in a T1 MRI scan, which is oil or fat based, the composition should contain more vitamin E oil. For an MRI sequence that exploits fat, such as Single Shot Fast Spin Echo T2-weighted, the composition should contain olive oil in the maximum amounts. And while the composition is effective in all imaging procedures, when the composition is to be used in x-ray and CT scans, which are based upon principles of opacity, it benefits to include more opaque components, such as hydroxyapatite or calcium carbonate.

While there is no particular method or order for mixing or preparing the composition of the current invention, two major points are useful in its preparation:
1. Mildly heating the fat and/or oil component helps in immediately creating a good suspension; and
2. The suspension should not be made to be completely homogenous, i.e., the oil and/or fat and the solvent should not be mixed and/or dissolved completely in each other, as that will only create a substance like mustard which show up only in some MRI sequences, depending on proportions.

### Imaging Procedures

While the composition of the current invention would be useful in any imaging procedure including those known and those to be developed in the future, the imaging procedures that are most widely used today in medicine are magnetic resonance imaging (MRI), x-rays, and x-ray computed tomography (CT).

The composition is also useful in non-medical imaging procedures such as those used in engineering applications.

Magnetic Resonance Imaging (MRI)

There are several MRI and scans for which the present invention is useful.

T1 and T2 sequences are used for visualization and quantification of fat and water respectively (Hu and Kan 2013; McMahon et al. 2011; Panigrahy et al. 2010). T1-weighted is an MRI made with pulse spin echo or inversion recovery sequence with short TR and TE to show contrast between tissues with different T1 values. T1 MRI results in an image with greater signal intensity from fat-containing tissues.

T1-weighted fluid-attenuated inversion recovery (FLAIR) is a refined T1 MRI, which provides good contrast between lesions, surrounding edematous tissue, and normal parenchyma at low field strengths and at acquisition times comparable to those of T1-weighted spin-echo imaging.

T2-weighed MRI is an image made with a sequence with long TR and TE to show contrast in tissues with varying T2 relaxation times. Water gives a strong signal in T2-weighted MRI. T2 can also be refined by using fluid-attenuated inversion recovery (FLAIR) pulse sequences to suppress fluid signals.

BRAVO is a high resolution, automated, rapid, 3D imaging technique that produces heavily T1-weighted isotropic images of the brain. It helps to visualize small and subtle lesions and has excellent T1 contrast between grey and white matter. The scan can be reformatted into any plane. It provides reduced scan time and minimized parallel imaging artifacts. BRAVO uses 3D IR-prepared FSPGR acquisition to produce isotropic T1-weighted volumes. (GE Healthcare BRAVO).

Diffusions tensor imaging or DTI MRI is a specialized MRI of the brain or spinal cord that evaluates neural pathways within the brain, brainstem, or spine, such as motor-skill controls and speech. DTI is based on the non-brownian movement of water molecules, the direction of which is determined by many factors, such as cell membranes, axonal membranes, and cytoskeletal structures. The anisotropic movement of water dominates in regions with high concentrations of axons. As a result, quantitative measurement of diffusion anisotropy can be an indicator of the integrity of cerebral white matter and thus, DTE is especially indicated for diseases causing axonal damage and demyelination.

Fractional anisotropy is a form of DTI using axonal fiber (white matter) tract clustering using a fractional anisotropy map. Anatomically distinct fiber tracts are drawn, then, white matter tractography is performed to interpolate paths (fiber traces) following the major directions of diffusion. Only the defined regions of interest are used to make traces. Fiber traces are then grouped using a pairwise similarity function (which uses the shapes of the fibers and their spatial locations). This method enables further estimation of anatomic connectivity between distant brain regions by finding fiber clustering that guides the separation of anatomically distinct fiber tracts. Several fiber tracts which would otherwise be difficult to define are separated using this clustering algorithm (O'Donnell et al. 2006).

Diffusion-weighted magnetic resonance imaging (DW-MRI or DWI) provides good quality images by allowing water movement and fat signal (defocused off-resonance) imaging with less distortion. Water movement can be imaged irrespective of direction. Because this technique is highly sensitive to the altered motion of water, it is used to diagnose many pathological conditions including acute ischemia, different types of cancers, intracranial infections, and autoimmune diseases (Schafer et al. 2011; Vandecaveye et al. 2010; Razek, 2010).

Single Shot Fast Spin Echo T2-weighted imaging (SSFSE) is a sub-second single-section T2-weighted technique that gives images with higher spatial resolution. A single excitation pulse is followed by a rapid train of refocused echoes, providing all the data needed for the image. Because the center of k-space is sampled within a fraction of a second, motion-induced artifact is nearly absent. SSFSE is used for most coronal localizer images, providing a rapid, motion-independent T2-weighted survey. Heavily T2-weighted SSFSE images are used for MR cholangiopancreatography (MRCP) and to characterize solid versus non-solid liver masses as well as to image fetal development and assess cystic lesions (Saleem 2014; Lefevre et al. 1998).

3D-FSGPR or fast spoiled gradient echo MRI is a very fast sequence which is relatively new and gives better soft tissue contrast. It helps to identify small cortical lesions (dysplastic ones which conventional 2D MRI identifies as normal), subtle structural abnormalities, and brain convolutions (sulci and hemispheric convexities). It helps to sample gray-white matter more symmetrically and reduce false-positives. It is useful in imaging aortic dissection, thoracic and abdominal aortic aneurysm, pulmonary embolus, carotid stenosis, and peripheral vascular disease. Rapid data acquisition times allows for imaging multiple temporal phases or multiple locations. This technique uses the suppression of fat signal, when the fat signal causes artifacts or otherwise obscures a tissue of interest. Furthermore, SPUR can be performed with 1-mm thin sections (Alikhanov et al. 2001; Al-Saeed et al. 2005)

Magnetic resonance angiography (MRA) and magnetic resonance venography (MRV) sequences have high significant clinical relevance and are routinely used. Pathological conditions in veins are investigated by MRV (Spritzer 2009), although duplex ultrasound and CT scanning are also utilized in identifying abnormalities such as obstructions. The same is true for arteries which are visualized best by sequences developed for oxygenated blood. MRA and CTA are replacing intra-arterial catheter angiography for the diagnosis of serious complications such as intra-cranial vascular diseases, because catheter angiography is invasive, requires in-patient admission and has been associated with neurological complications.

As shown in FIGS. 1-6, the composition of the current invention shows with contrast and definition in all types of MRI images.

X-Rays

Human body or other structures differentially attenuate x-rays and create shadows that are captured by x-ray-sensitive detectors, a method that has been used since 1896 following its discovery by Roentgen (Seibert 2004). The transmitted fractions of the x-ray beams, captured by the detectors are now widely used to study objects hidden to the naked eye and they have been especially useful in medicine, such as orthopedic and pulmonary medicine. However, since the transmission and attenuation depends on the opacity of the imaged objects, it is still difficult to easily image and know the exact shape of objects that are relatively transparent, for instance skin compared to hone. If one can use the composition of the present invention to achieve this goal, then there will be no need for another kind of scan (say MRI) for further analysis/understanding of the object (body part) under study.

As shown in FIG. 7, even under low intensity image quality conditions, the composition can be visualized.

X-Ray Computed Tomography (CT) Scans

Following the success of x-ray imaging, instruments which recorded attenuation of x-ray beams around 180 degrees after they passed through the imaged object, it was possible to get body cross-section images (Robb 1982; Trattner et al. 2014). This method is called x-ray computed tomography (CT). It too can benefit from a compound that can outline X-ray transparent objects.

As shown in FIGS. 8 and 9, the composition can be utilized in CT scans.

### Uses for Compound/Composition

Since MRI, CT and x-rays are the best non-invasive tests to diagnose and treat various medical conditions, they are widely used. Thus, the compound or composition of the current invention will have many uses and applications. It will be understood by those of skill in the art that the composition is useful for all types of imaging procedures, those known now and those to be developed in the future.

Generally the method of using the composition comprises applying the composition to an external or internal surface, such as external biological tissue of the subject, e.g., skin, or internal biological tissue that may be exposed during testing, e.g, dura, or an internal or external non-naturally occurring structure, e.g., an electrode, or an internal naturally occurring structure, i.e., organ, that is not visible on an image produced by an imaging procedure. The composition solves this problem by making the surface or structure visible on the image. The composition of the present invention is both flexible and able to conform to any shape of the external or internal body, as well as able to adhere well to skin and hair.

A further embodiment of the present invention is a method of using the image produced by the composition to localize an internal structure of interest. The external or internal surface or structure to which the compound is applied to make visible on an image, can have a known spatial relationship with the internal structure of interest. After the subject is imaged using an imaging procedure, the composition will be seen on the image allowing the surface or structure to be seen on the image and the skilled practitioner will be able to see the internal structure in relation to the external or internal surface or structure via the composition which now can be visualized in the scan.

The method can be performed by applying the composition of the present invention to an external or internal surface, either naturally occurring or non-naturally occurring, such as biological tissue, such as skin or dura, or tape, or by applying the composition of the present invention to an external or internal structure, either naturally occurring or non-naturally occurring, such as a lead, electrode, monitoring device, tape, marker, or prosthetic, that is at a known external or internal location on the body of the subject. After the scan, the composition will show an image of external or internal surface or structure that can be used to localize the internal structure of interest.

The method can also be performed by placing the composition into a vessel or device such as a tube, and placing the device on or attaching it to the external surface of the body of the subject. This can be accomplished also by attaching the tubes to a helmet or cap as shown in Example 4. Alternatively, the vessel or tube or capsule can be inserted internally into the subject. The subject is then scanned and the composition is seen on the resulting images and can be used to localize the internal structure of interest.

As can be seen, there are numerous applications for use of the marker compound/composition in MRI, CT scans and x-rays, more than the specific examples that are set forth below.

Many clinical interventions, such as targeted drug delivery and surgery, require localization of various body parts or structures. One specific use for the composition of the present invention is for facial surgery, such as plastic surgery procedures. In this procedure, the composition would be applied to external areas of the face to localize the structures of interest internally. After the scan, the precise location for incision on the face would be shown by the image of the composition.

With increased ease, power and accessibility of MRI, especially the high resolution of T1-weighted structural MRI, there has been a growing interest in using this technology to study brain structure, function, development, and pathologies. In brain surgery, a precise craniotomy, which gives perfect access to the brain area of interest is needed and dependent on correctly establishing the relationship between head landmarks and structural MRI scans. By applying the composition to the external part of the head which have a known spatial relationship with the underlying internal structure, the underlying structure can be precisely located for incision.

Another specific use for the composition is with leads or electrodes, either in a diagnostic procedure on a human or in research involving non-human subjects. For instance, a patient in a sleep or epilepsy diagnostic or research clinic could have MRI-safe EEG leads put on the external surface of the brain and the leads could be localized to the underlying brain areas by applying the composition to the leads. The composition can even be applied in varying amounts to the leads, depending on the practitioner's interest or suspicions as to disease. For example, a larger amount of composition could be placed on a lead where the practitioner suspects an epileptic focus.

Additionally, the electrically conductive gel normally embedded in connector terminals of EEG, EKG and other leads, even surgical (laparoscopy) and imaging wands can be made with the composition of the invention in them, making 3D visualization possible.

In another general laboratory research practice, non-human primate skulls are routinely covered with a dental acrylic, either to cover a craniotomy or to secure a head-posting structure. Being an ‘inert’ structure, the dental acrylic (cement) cannot be seen in any MRI scan. Using the composition however, its outlines can now be visualized making it possible to use it in future surgeries, or following up of any process inside the brain.

Another common practice to guide surgeries in humans, non-human primates and other animals, is registering the head to acquired MR images in reference to a 3D position sensor. To do this, before surgeries are performed, MRIs are routinely acquired with fiducial markers fixed to the head (headpost in animals) (for instance using markers from Brainsight, Rogue Research, Quebec, Canada). Then, during the surgical procedure, the fiducial markers are reattached, and using the MRI, specific brain structures are registered and targeted. While this procedure is highly useful, it is sometimes possible to lose the marker signals in the initial scans, either due to the sequence being not the right one, or possibly because of marker age. As shown in Example 6 and FIG. 6, when BRAVO and T1 scans were performed using the marker alone, no image was seen. After the compound of the invention was applied to the markers, they were visible on both scanned images.

This composition can also be used for specialized sequences. For instance, the integrity of brain white matter is quantified and characterized by diffusion tensor imaging (DTI) (Chanraud et al. 2010). This sequence is based on the principle that MRI is fundamentally imaging water protons based on movement of water molecules which are usually random (anisotropic) except in instances where they are bound by scaffolds such as fibers where they are forced into axial or longitudinal diffusion. This sequence thus allows one to study brain connectivity in addition to integrity characterization. Accordingly, the composition of the present invention can be incorporated into artificial fibers with predetermined architecture based on the need and application environment. “Artificial fibers” specifically means viscous chemicals which have a locally non-random connectivity pattern giving directionality to water molecule movement during MRI.

The composition can be encapsulated in a sterile agent and place internally as a marker after surgery, as for example following cancerous tissue contraction due to intervention.

The composition can also be used in developing and future technologies, such as hybrid PET/MR systems (Runge 2013; Disselhorst et al. 2014). Moreover, anatomic, functional and molecular imaging and integration of biological data with medical imaging is emerging (Huang and Shih 2014) and the composition of the present invention can be used to register high-resolution internal anatomical images with external markers.

Additionally the composition of the present invention can be used for material science applications such as building other structures, such as artificial ceramic hips for purposes such as doing finite element analysis in artificial car crashes.

### Examples

The present invention may be better understood by reference to the following non-limiting examples, which are presented in order to more fully illustrate the preferred embodiments of the invention. They should in no way be construed to limit the broad scope of the invention.

### Example 1-Materials and Methods

Composition/Compound

The composition or compound used in the following examples was made with the following ingredients (percent by volume):

Water (formulation 1) or povidone-iodine (Betadine®) (formulation 2):40%

Vitamin E oil: 20%

Purified butter: 10%

Olive Oil (slightly warmed) 10%

Ground thyme: 7.5%

Ground cloves: 2.5%

Hydroxyapatitc (i.e., calcium phosphate) or calcium carbonate: 5%

Protein powder: 5%

Few drops of green food coloring

Animal Procedures

All animal procedures were done per Columbia University Medical Center animal research regulations and the Michael E. Goldberg laboratory protocols.

Monkeys (Macaca radiata) were anesthetized in their home cage with Ketamine (10-20 mg/kg) injection intramuscularly. Most of the monkeys remain under anesthetic for the duration of the scanning by just this drug alone.

For the MRI procedure, the anesthetized monkeys were transported to the Columbia University Department of Radiology. In the facility, after their head was stabilized with ear bars, the monkeys were placed into the scanner chamber, and their heads fitted inside a 16-inch head coil. Monkeys remain anesthetized during the MRI procedure, if needed, with a supplemental injection of Ketamine. Since the procedure takes only 30-50 minutes, the first anesthesia injections were generally effective in maintaining stillness inside the machine.

MRIs of brains were acquired on a 1.5 T GE Medical Systems Signa Excite Scanner, with typical parameters as shown in Table 1.

After being transported back to their cages, monkeys were allowed to completely recover from the effects of the anesthesia.

Assuming all monkeys were positive for Hepatitis B Virus, all personnel working on this procedure follow sterile techniques described in our protocol including wearing masks, gowns or aprons as well as boots or protective shoe covers, and goggles. All MRI machine surfaces are cleaned and disinfected with AHP as described.

AHP here refers to “Accelerated Hydrogen Peroxide Surface Disinfectant” (sold as 7% Virox 5 Concentrate, Virox 5 Ready-To-Use and/or Virox 5 Wipes, 7% PerCept Concentrate, PerCept RTU or PerCept Wipes, 7% Accel Surface Cleaner Disinfectant Concentrate, Accel RTU or Accel Wipes) and 0.5% Accelerated Hydrogen Peroxide Tuberculocidal Surface Disinfectant (sold as Aced TB TRU or Accel TB Wipes).

1. Preparation of solution—Pre-mix and label from a controlled location 7% AHP Concentrate at a ratio of 1:16 (0.5% AHP).

2. Place mixed solution in either a labeled—flip top 1 Litre bottle or a small hand bucket.

3. AHP RTU is ready to use (0.5% AHP).

4. AHP Wipes are ready to use (0.5% AHP).

Surfaces are cleaned repeatedly by reapplying the AHP Solution and allowing for a 5-minute contact time.

MRI Specifications

The specifications for the MRI procedures performed in the some of the Examples are set forth in Table 1.

TABLE 1
MRI Specifications

| | | Echo | Repetition | Inversion | | Field of | | |
|---|---|---|---|---|---|---|---|---|
| | Acquisition | Time | Time | Time | Thickness | view | Matrix | Pixel |
| Series | type | (TE) (ms) | (TR) | (TI) | (mm) | (FoV) | size | spacing |
| T1 | 3D | 4 | 34 ms | 0 | 1 | 120 * 120 | 256 * 256 | 0.47 × 0.47 mm |
| T2 | 2D | 97.272 | 5200 | 0 | 2 | 120 * 120 | 256 * 256 | 0.47 × 0.47 mm |
| Flair | 2D | 120.64 | 10002 | 2200 | 2 | 120 * 120 | 224 * 256 | 0.47 × 0.47 mm |

### Example 2—Comparison of the Composition to Individual Ingredients and Prior Art Markers

The composition of Example 1 (formulation 1), the composition plus gadolinium, ingredient #1 (olive oil), ingredient #2 (butter), ingredient #3 (water), gadolinium (Gd), toothpaste, toothpaste plus gadolinium (Gd), and vitamin E tablet were separately packed into different compartments of a small multi-pocketed pouch, and scanned using T1, T2, and Flair MRI on 1.5 GE Medical Systems Signa Excite Scanner, using the specifications set forth in Table 1.

As seen in FIG. 1, the composition of the present invention (labeled “Mix”) was the only composition that showed up on all three types of scans. This result showed that the marker paste composition of the present invention solved the problem of the prior art compound as it showed up on all three types of scans used.

### Example 3—Use of Composition of the Present Invention in MRI Scans of Monkey Brains

Using the composition of Example 1 (formulation 1) and the procedures for monkey testing set forth in Example 1, MRI scans of the monkeys' brains were performed. BRAVO, T2 FSE, T1 FLAIR and T2 FLAIR were performed. FIG. 2B shows the placement of the acrylic and composition in the monkey.

As shown in FIG. 2A, the composition (dark arrows) is seen on top of the dental acrylic (white arrows) in each of the four types of scans.

These results showed that the composition of the present invention can be used in actual scans of living organisms. It also showed that it can be used to outline and visualize structures that would be useful to visualize during MRI, such as recording chambers, electrodes, dental and other acrylics, surgical tape, grids and other structures that are not a part of the subject's body.

Additionally, the composition of the present invention can be put over any body part of the subject's body, including skin on the patient's head, neck, chest, abdomen, and limbs.

### Example 4—Use of the Composition in MRI Scans Using an EEG Cap Mimic and Several MRI Types

The composition of Example 1 (formulation 1) was filled into small vinyl tubing which was glued to a plastic helmet that mimics an EEG cap, as shown in FIG. 3. This type of wearable landmark was imaged overlaying a mimic of body part, the head. Various types of MRI scans outlined above were performed of the plastic cap of FIG. 3 at the NYSPI MRI center using a GE Medical Systems Discovery MR750 3-T scanner.

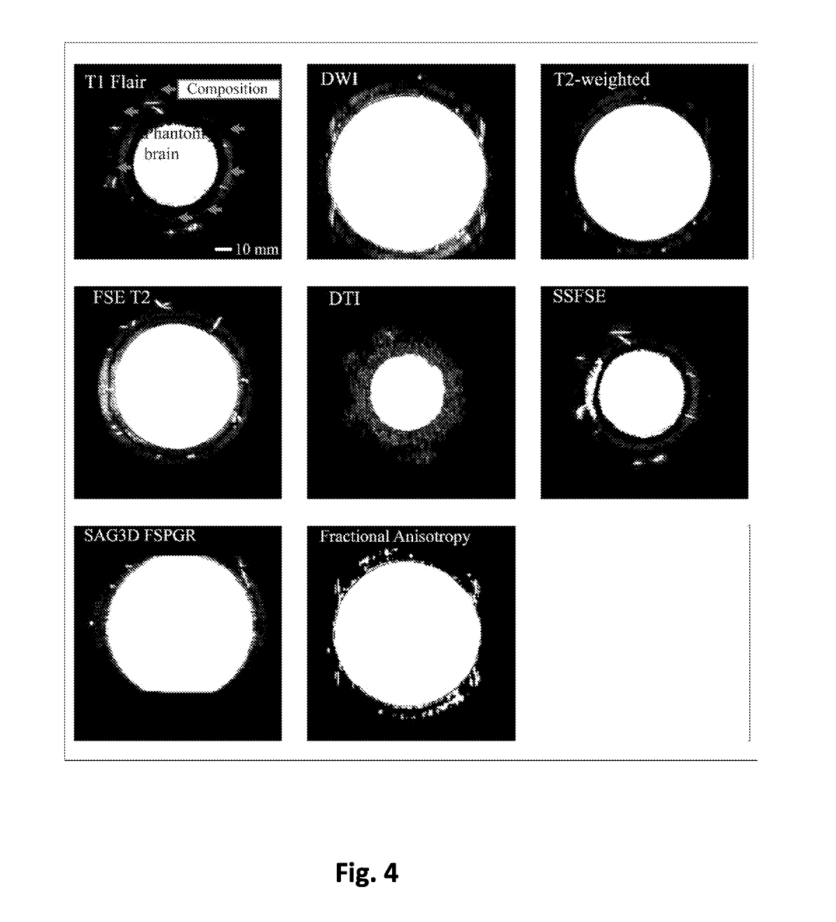

As shown in FIG. 4, the tubes containing the compound were seen in all of the different types of MRI scans, including T1 FLAIR, DWI, T2-weighted, FSE T2, DTI, SSFSE, FSPGR, and fractional anisotropy.

### Example 5—Use of the Composition for Additional MRI Imaging Applications

To further show the use of the composition for imaging using T1 and T2, and to show its use for other sequences, including magnetic resonance angiogram (MRA, blood vessel imaging for arteries) and magnetic resonance venography (MRV, blood vessel imaging for veins), additional scans were performed using the composition, with and without dental acrylic, using a scan of a brain for comparison at the NYSPI MRI center using a GE Medical Systems Discovery MR750 3-T scanner.

Formulation 1 and formulation 2 of the composition (Example 1) was used with and without dental acrylic ((A)—formulation 1 plus dental acrylic, (B)— formulation 2 plus dental acrylic, (D)—formulation 1 alone, and (E)—formulation 2 alone). Dental acrylic alone (C) was also used. All of the preparations were put in surgical syringes for stability during scans and ease in visual comparison.

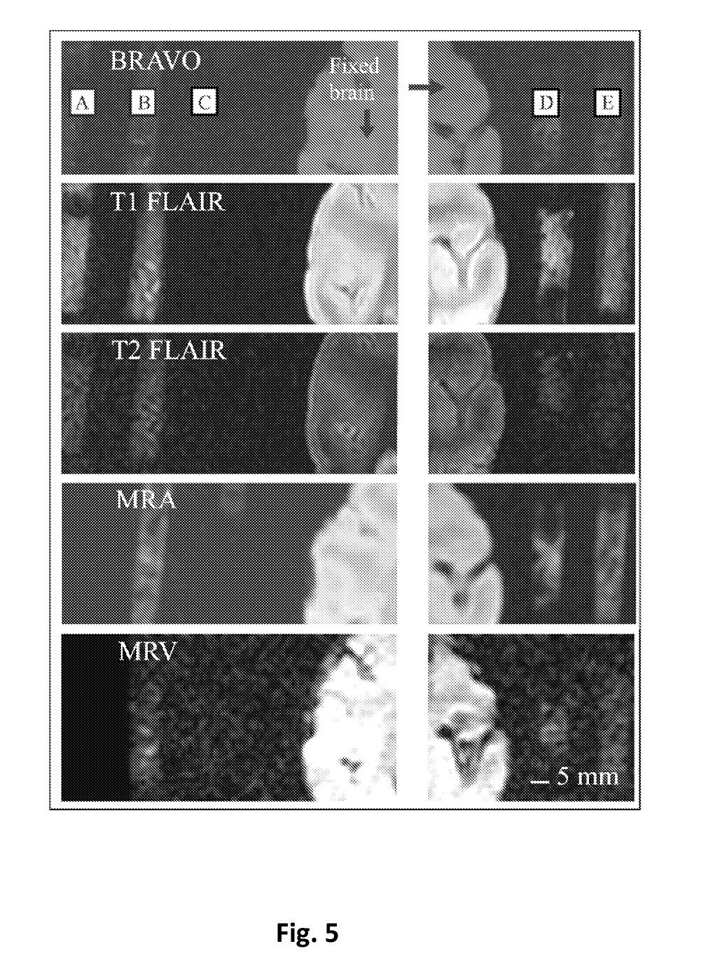

As can be seen in FIG. 5, the composition showed with contrast and definition, patterns similar to the human brain. The dental acrylic alone did not show contrast or definition.

### Example 6—Use of the Composition for MRI Imaging of Markers

Fiducial markers (Brainsight Rogue Research, Quebec, Canada) were fixed to the head of monkeys using the procedures for the product. Using the procedures for monkey testing set forth in Example 1, MRI scans of the monkeys' brains were performed (T1 and BRAVO) at the NYSPI MRI center on a GE Medical Systems Discovery MR750 3-T scanner. As seen in the left hand panels of FIG. 6, the markers were not visible on either scan.

When the compound of Example 1 (formulation 1) was painted on the markers, they became visible in both the T1 and BRAVO scans (right hand panels of FIG. 6).

### Example 7—Use of the Composition in X-Rays

The composition was x-rayed using the same view of the machine as other structures such as a fixed brain (as described before), stainless steel rod (A), and microelectrodes (G) and (H)) (FIG. 7). The x-rays were performed at New York Presbyterian at Columbia University Medical Center, using GE Thunder Platform.

Formulations 1 and 2 of the composition (as set forth in Example 1) were used alone and mixed with dental acrylic in the x-rays ((B)— formulation 1 plus dental acrylic, (C)—formulation 2 plus dental acrylic, (E)—formulation 1 alone, and (F)— formulation 2 alone). Dental acrylic alone was also used (D). The five preparations were put in surgical syringes for stability during scans and ease in visual comparison. A stainless steel guide tube (2 mm diameter) (A) was also imaged by x-ray.

In addition, the composition was x-rayed in 0.5 mm wide plastic guide-tubes ((I)—formulation 1 and (J)—formulation 2) and compared to microelectrodes (G) and (H).

FIG. 7 shows the results of the x-ray, with the same scan being presented with increasing contrast from top to bottom panels. The x-ray scan results showed that even under low intensity image quality conditions, the composition can be visualized in the images.

### Example 8—Use of the Composition in CT Scans

The composition was used in a CT scan using the same view of the machine as other structures such as a fixed brain (as described before) stainless steel rod (A), and microelectrodes (G) and (H)) (FIG. 8). The images captured in a Siemens CT scanner at the PET Center at Columbia University Medical Center Department of Radiology.

Formulations 1 and 2 of the imaging composition (as set forth in Example 1) were used alone and mixed with dental acrylic in the x-rays ((B)— formulation 1 plus dental acrylic, (C)— formulation 2 plus dental acrylic, (E)—formulation 1 alone, and (F)— formulation 2 alone). Dental acrylic alone was also used (D). The five preparations were put in surgical syringes for stability during scans and ease in visual comparison. A stainless steel guide tube (2 mm diameter) (A) was also imaged by x-ray.

In addition, the composition was scanned in 0.5 mm wide plastic guide-tubes (I-formulation 1 and J—formulation 2) and compared to microelectrodes (G) and (H).

FIG. 8 shows the results of the CT scan, with the same scan being presented with increasing contrast from top to bottom panels. The images captured in the CT scanner showed that the composition was visible and had well-defined edges.

As there is a growing concern about radiation exposure and there are several approaches to reduce dose (Trattner et al. 2014), the composition was scanned with both high and low dose conditions (FIG. 9) and the results showed that there were no critical and visible losses in image quality.

### REFERENCES

Al-Saeed et al. (2005) Australas Radiol. 49(3):214-7
Alikhanov et al. (2001) Vesln. Rentgenol. Radiol. (2):9-16
Chanraud et al. (2010) Neuropsychol. Rev. 20: 209-225
Disselhorst et al. (2014) J. Nucl. Med. 55 (Supplement 2): 2S-10S
GE_Healthcare. BRAVO: GE Healthcare; 2012
Hu and Kan (2013) NMR Biomed. 26(12):1609-29
Huang and Shih (2014) Biomed. Res. Int. 2014: 365812
Lefevre et al. (1998) J. Radiol. 79(5):415-25
McMahon et al. (2022) J. Orthop. Sports Phys. Ther. 41(11):806-19
O'Donnell et al. (2006) Am. J. Neuroradiol. 27(5):1032-6
Panigrahy et al. (2010) Semin. Perinatol. 34(1):3-19
Razek (2010) J. Comput. Assist. Tomogr. 34(6):808-15
Saunders et al. (1990) Exp. Brain Res. 81(2): 443-446
Schafer et al. (2011) Magn. Reson. Imaging Clin. N. Am. 19(1):55-67.
Semework (2010) 26th Southern Biomedical Engineering Conference SBEC 2010, IFMBE Proceedings 32:493-49
Spritzer (2009) Perspect Vasc. Surg. Endovasc. Ther. 21:105-116
Trattner et al. (2014) J. Am. Coll. Radiol. 11(3): 271-278
Van Essen et al. (1998) Proc. Natl. Acad. Sci. USA 95(3): 788-795
Vandecaveye et al. (2010) Neuroradiology 52(9):773-84

1. A composition comprising vitamin E, a solvent, and fat, wherein the composition is in a form suitable for the topical application to a subject undergoing an imaging procedure, and the composition can be visualized by the imaging procedure.

2. The composition of claim 1, wherein the imaging procedure is selected from the group consisting of magnetic resonance imaging, x-ray, and x-ray computed tomography imaging.

3. The composition of claim 1, wherein the vitamin E is present in the composition in an amount of about 15 to 60% by volume.

4. The composition of claim 1, wherein the vitamin E is present in the composition in an amount of about 20 to 30% by volume.

5. The composition of claim 1, wherein the vitamin E is present in the composition in an amount of about 15 to 20% by volume.

6. The composition of claim 1, wherein the solvent is selected from the group consisting of water and povidone-iodine.

7. The composition of claim 6, wherein the water is selected from the group consisting of purified, distilled, double distilled, and deionized.

8. The composition of claim 1, wherein the solvent is present in the composition in an amount of about 25 to 75% by volume.

9. The composition of claim 1, wherein the solvent is present in the composition in an amount of about 30 to 45% by volume.

10. The composition of claim 1, wherein the fat is selected from the group consisting of purified butter, vegetable shortening, coconut oil and lard.

11. The composition of claim 1, wherein the fat is present in the composition in amount of about 15 to 60% by volume

12. The composition of claim 1, wherein the fat is present in the composition in an amount of about 20 to 30% by volume.

13. The composition of claim 1, wherein the fat is present in the composition in amount of about 5 to 10% by volume

14. The composition of claim 1, further comprising at least one additional component chosen from the group consisting of oil, an emulsifying agent, an iron containing substance, a manganese containing substance, calcium carbonate, calcium phosphate, protein powder, and food coloring.

15.-32. (canceled)

33. The composition of claim 1, wherein the composition is in the form of a capsule, paste, tablet or solution.

34. The composition of claim 1, wherein the composition is placed into a device or vessel that is contacted with or placed on or attached to the external surface of the body of the subject undergoing an imaging procedure.

35. The composition of claim 1, wherein the composition is loaded into a machine for real time visualization and orienting during surgery.

36. A composition comprising vitamin E, a solvent, purified butter, olive oil, thyme, cloves calcium phosphate or calcium carbonate, and protein powder, wherein the composition is in a form suitable for the topical application to a subject undergoing an imaging procedure, and the composition can be visualized by the imaging procedure.

37. A method of using the composition of claim 1, comprising:
a. applying the composition of claim 1 to a surface or structure that is normally invisible on an image produced by an imaging procedure;
b. performing an imaging procedure; and
c. visualizing the composition applied to surface or structure on the image from the imaging procedure.

38. (canceled)

39. (canceled)

40. A method of using the composition of claim 36, comprising:
a. applying the composition of claim 36 to a surface or structure that is normally invisible on an image produced by an imaging procedure;
b. performing an imaging procedure; and
c. visualizing the composition applied to the surface or structure on the image from the imaging procedure.

41.-42. (canceled)

43. A method for localizing an internal structure in relation to an external or internal surface or structure of a subject undergoing an imaging procedure comprising:
a. applying the composition of claim 1, to the external or internal surface or structure of the subject, wherein the external or internal surface or structure can have a known spatial relationship with the internal structure of the subject;
b. imaging the subject with the imaging procedure;
c. visualizing the composition applied to the external or internal surface or structure on an image produced by the imaging procedure; and
d. localizing the internal structure in relation to the external or internal surface or structure of the subject from the location of the composition on the image.

44.-51. (canceled)

52. A method for localizing an internal structure in a subject undergoing an imaging procedure, comprising:
a. preparing a device or vessel that contains the composition of claim 1, and wherein the device or vessel can be placed on or contacted with or attached to an external or internal surface or structure of the subject;
b. imaging the subject with the imaging procedure;
c. visualizing the composition on an image produced by imaging procedure; and
d. localizing the internal structure of the subject in relation to the device or vessel from the location of the composition on the image.

53.-58. (canceled)

59. A method for localizing an internal structure in a subject undergoing surgery using an orienting or intraoperative machine, comprising:
a. loading the composition of claim 1, into the machine, wherein the composition can be visualized by imaging;
b. performing the surgery on the subject; and
c. localizing the internal structure using the image of the composition.

60.-62. (canceled)
